(19) United States
(12) Patent Application Publication
(10) Pub. No.: US 2020/0108391 A1
(43) Pub. Date: Apr. 9, 2020

(54) Device and Method For The Continuous Trapping of Circulating Tumor Cells

(71) Applicant: University of Twente, Enschede (NL)

(72) Inventors: Michiel Stevens, Deventer (NL); Arjan G.J. Tibbe, Deventer (NL); Joska Johannes Broekmaat, Enschede (NL); Frederic Thomas NIJSINK, KD Rijssen (NL); Leon W.M.M. Terstappen, Amsterdam (NL); Guus van Dallum, Herzogenrath (DE)

(73) Assignee: University of Twente, Enschede (NL)

(21) Appl. No.: 16/500,948

(22) PCT Filed: Apr. 13, 2018
(86) PCT No.: PCT/EP2018/059607

§ 371 Date: Oct. 4, 2019

Related U.S. Application Data
(60) Provisional application No. 62/485,414, filed on Apr. 14, 2017.

(51) Int. Cl. B01L 3/00; G01N 33/543; G01N 33/574

(52) U.S. Cl. CPC B01L 3/502761; B01L 3/502753; G01N 33/54326; G01N 33/574; B01L 2400/043; B01L 2200/0652; B01L 2300/088; B01L 2300/06; B01L 2300/0829

(57) ABSTRACT

The present invention describes a method and device for improving the capture and interrogation of a rare cell population in a biological fluid such as in circulating tumor cells (CTC) in blood or Diagnostic Leukapheresis. ReFLECT-CTC is designed to capture CTC in a continuous fashion and interrogate isolated individual CTC. ReFLECT-CTC has the advantage of sampling large volumes of a biological sample which is especially useful in assessing the heterogeneity of a CTC population. The invention has application in cancer diagnostics where assessing tumor cells found in individual cancer patients will determine which drugs are most likely to be effective for an individual.

### CROSS REFERENCE TO RELATED APPLICATIONS

This application is based on and claims priority to U.S. Provisional Application U.S. 62/485,414, filed on 14 Apr. 2017, the disclosures of which are herein incorporated by reference.

### BACKGROUND

### Field of Invention

The present invention relates to the isolation of tumor cells and tumor derived extracellular vesicles. The tumor cells can be expanded and DNA, RNA and proteins can be extracted from the individual tumor cells or their secreted products analyzed to enable full characterization of the cancer cells. More specifically, the present invention relates to a method and a device for the isolation and characterization of tumor cells from large blood volumes.

### Description of Related Art

The presence of tumor cells enumerated with the CellSearch system in 7.5 ml of blood from cancer patients is associated with poor prognosis. Elimination of circulating tumor cells (CTC) after 3-5 weeks of therapy indicates an effective therapy whereas their continued presence indicates a futile therapy. These observations have evoked the interest of researchers and clinicians worldwide and resulted in a large number of new approaches to capture these CTC and extract information from this “liquid biopsy.” We have however shown that in the majority of patients there are insufficient tumor cells present to represent a biopsy and predicted that an increase in a blood volume to 1-2 liter is needed to isolate a sufficient number of CTC in all metastatic cancer patients. Leukapheresis has been introduced to increase the blood volume for the isolation of CTC. In this procedure, the proven technique of leukapheresis is used to collect an amount of mononuclear cells (MNC) equivalent to 1-2 liters of blood and an equivalently higher number of CTC. These observations have been confirmed recently by the EUFP7 CTCTrap consortium and are currently being evaluated in Non Small Cell Lung Cancer (NSCLS) in the EU IMI CANCER-ID consortium. The technologies evaluated to extract CTC from the leukapheresis products can however only process 1-10% of sample. Methods to deplete leukocytes using targeting antigens specific for leukocytes or by size/density based methods such as filtration were accompanied by large CTC losses and/or could handle only small volumes. Most successful was the use of the CellSearch system that uses Epithelial Cell Adhesion Molecule (EpCAM) coated ferrofluids, but only 2 ml of Diagnostic Leukapheresis (DLA) product can be processed.

Accordingly, current circulating tumor cell enumeration and isolation techniques only use a small quantity of patient blood to assess the amount of CTC in order to gain insight in the makeup of these CTC. The use of larger quantities for processing will lead to a larger number of CTC available to probe for the presence of treatment targets and will increase the proportion of patients where CTC are detected. In addition, more insights can be obtained into the heterogeneity of CTC through the availability of more tumor cells and the ability to isolate a single CTC. Having this information results in more insights obtained not only on the effectiveness of therapies administered to patients but also on the relation between the heterogeneity of the tumor and metastasis on the one hand and the heterogeneity of the CTC population on the other.

Treatment decisions are difficult to make based on a single digit number of CTC or a representation of only a single sub-clone. The ability to effectively obtain a liquid biopsy using the device disclosed herein will significantly improve the treatment of cancer patients and will on the one hand reduce the economic burden of cancer therapies by creating the potential to only provide therapies that will be effective and on the other hand will increase the wellbeing of the patient by avoiding therapies that are not effective.

Therefore, it has been determined that there is a need for a means to isolate and characterize tumor cells and other rare cells from large blood volumes.

### SUMMARY

In the majority of cancer patients, the frequency of Circulating Tumor Cells in a single tube of blood is not enough to fully characterize the cancer. Although a larger number of tumor cells can be obtained through leukapheresis, the technologies available today can only extract CTC from a small fraction. The leukapheresis product for a Diagnostic Leukapheresis is typically 40 ml containing approximately 25×10⁸ mononuclear cells (or approximately 2 liters of blood), of which currently only 2 ml can be processed. Here we introduce ReFLECT-CTC, a novel technology that utilizes a fixed amount of epithelial cell specific antibody-labeled ferrofluids to capture and isolate tumor cells from DLA product. These ferrofluids are contained within a disposable cassette by magnets, which allow continuous passage of the sample while containing the ferrofluids and the captured cells labelled with ferrofluids. After the sample has passed through the cassette, the tumor cells and residual leukocytes captured onto the antibody labeled ferrofluids are flushed out of the cassette. These cells are then for example placed on self-sorting microwells for identification of the tumor cells, their isolation as single cells for further characterization and probing with the most effective drugs. In addition to the isolation and interrogation of CTC, the process can be applied to other rare events and applied to other diseases.

Accordingly, the invention is designed to capture CTC in a continuous fashion thereby allowing for the improved capture and interrogation of CTC and offering an improved means to assess tumor cells for individual cancer patients in order to determine which drugs are most likely to be effective for an individual patient. Although tumor derived proteins, RNA and DNA in blood can provide an indication of which therapy is suitable, the actual tumor cells are needed to assess the heterogeneity of the cancer cells with respect to the therapeutic targets and to actually test the drugs on the tumor cells. In the majority of cancer patients, the number of tumor cells that can be isolated from a tube of blood is however not sufficient to select the optimal therapy. The methods disclosed herein enable the isolation and characterization of tumor cells from larger blood volumes for improving therapy.

The invention provides, in one aspect, a device for capturing a target cell population in a biological fluid comprising: (a) a container having an incubation chamber with an inlet and outlet for inflow and outflow of the biological fluid containing a target cell population; (b) a multiplicity of unbound cell specific antibody-labeled ferrofluids contained in the incubation chamber; and (c) a magnetic field to position the unbound cell specific antibody-labeled ferrofluids for binding to the target cell population and to retain both bound and unbound ferrofluids within said device, whereby flow of said biological fluid through said device may be continued indefinitely, in order to capture a desired quantity of the target cell population.

In another aspect, the invention provides a method for capturing cells from a target population in a biological fluid sample comprising: (a) flowing a biological fluid through an incubation chamber having an inlet and outlet; (b) exposing a multiplicity of unbound cell specific antibody-labeled ferrofluids in the incubation chamber to the biological fluid containing the target population; (c) positioning the unbound cell specific antibody-labeled ferrofluids with a magnetic field to bind to the target cell population in the biological fluid; (d) retaining both bound and unbound ferrofluids by means of a magnetic field; and (e) continuing the flow of said biological fluid until a desired quantity of the target cell population has been captured.

In a further aspect, the invention provides a method for analyzing the heterogeneity of a circulating tumor cell (CTC) population in a biological fluid sample comprising: (a) flowing a biological fluid through an incubation chamber having an inlet and outlet; (b) exposing a multiplicity of unbound CTC specific antibody-labeled ferrofluids in the incubation chamber to the biological fluid containing the CTC population; (c) positioning the unbound CTC specific antibody-labeled ferrofluids with a magnetic field to bind to the CTC cell population in the biological fluid; (d) retaining both bound and unbound ferrofluids by means of a magnetic field; and (e) continuing the flow of said biological fluid until a desired quantity of the CTC population has been captured, wherein the flow of the biological fluid containing the CTC population is in a quantity sufficient to analyze heterogeneity of the CTC cell population.

The above-described and other features and advantages of the present disclosure will be appreciated and understood by those skilled in the art from the following detailed description, drawings, and appended claims. In the following description, the invention is described in detail, by way of example only.

### BRIEF DESCRIPTION OF THE FIGURES

FIG. 1 Schematic representation of the ReFLECT-CTC principle.

FIG. 2 The configuration for a Rolling Ferrofluids design.

FIG. 3 Prototype for the Rolling Ferrofluids device.

FIG. 4 The configuration for a Flow Switching design.

FIG. 5 The configuration for a Chamber Containment design. Shown in Panel (a) are four stages of CTC binding and capture. Panel (b) shows the continuous flow by cycling four chambers through the four stages in sequence.

FIG. 6 The configuration for a Curtain design.

FIG. 7 Schematic representation showing the seeding of single cells within individual microwells. Panel A shows the initial entry of the target cell into the microwell. Panel B shows the same microwell with the flow diverted because of the occluded pore. Eventually more cells block the pores of the individual microwells as shown in Panel C. Panel D represents the completion of the seeding where individual cells are seeded within individual microwells.

FIG. 8 shows a representation of the sequence of steps in cell identification (Panel 1), isolation (Panel 2), and cell culture (Panel 3).

### DETAILED DESCRIPTION OF INVENTION

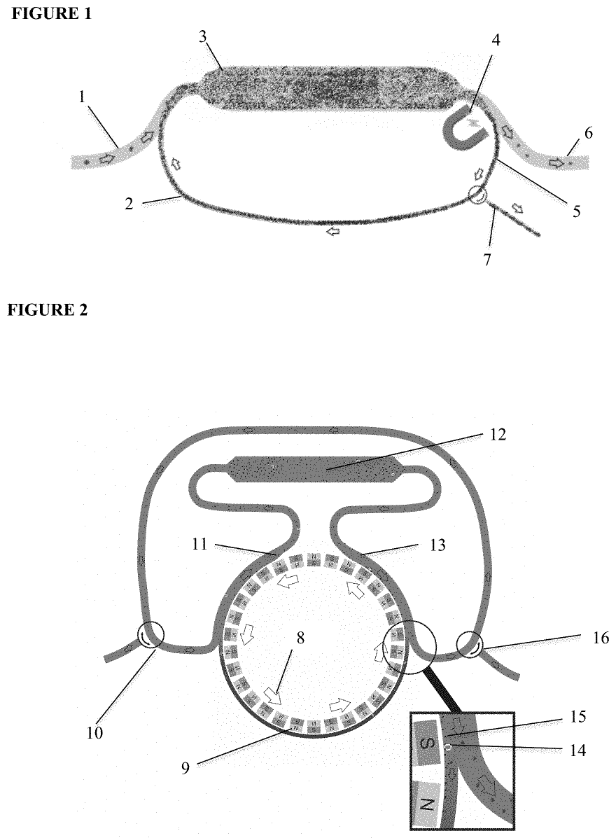

The present invention provides for the passage of large sample volumes in a continuous CTC capture and offers a solution to the insufficient volume of blood obtained for CTC analysis from a single tube of blood. This general process is classified herein as the ReFLECT principle. The general concept as illustrated in FIG. 1 shows a continuous CTC capture from diagnostic leukapheresis (DLA) or any other bodily fluid using antibody coated ferrofluids which are re-used after capturing CTC in an incubation chamber, such as in a cassette having a confined space. Fluid (1) is brought in contact with the ferrofluids (2) and enters the first phase of the process where incubation of a first fluid volume with the antibody-coated ferrofluids occurs (3). The fluid volume next enters a magnetic field (4) where free and bound antibody-coated ferrofluids (5) are diverted from the fluid volume (6). The diverted ferrofluids contain captured CTC bound to a population of antibody-coated ferrofluids along with a population of unbound ferrofluids. These ferrofluids are recirculated back into the incubation space for further interaction with another fluid volume. The process is continued until the desired volume of fluid has been sampled. The captured target cell can then be flushed from the cartridge for further analysis (7). The heterogeneous subsets of CTC, found though this process, represent the relevant sub clones found in metastasis.

The amount of CTC gathered by the ReFLECT principle is sufficient to assess the heterogeneity of the CTC population after single cell isolation and individual characterization. In one embodiment, EpCAM antibodies from the hybridoma VU1D9 are used. Other antibodies or binding agents are also considered as long as they do not react with blood cells.

For identification of specific cell types in a heterogeneous cell suspension such as blood, monoclonal antibodies recognizing specific targets are commonly used. The proportion of antibodies specific for a certain cell type that actually bind to the target cells in such a reaction is extremely low and for cells present in a low concentration one can potentially completely miss the fraction of antibody bound to the target cells. The reaction is mainly driven by the concentration of the antibody and the affinity for its target. When one passes a cell suspension containing a rare cell type through a solution of antibodies and gives it sufficient time to react with its target one could in principle label the rare cells without significantly changing the antibody concentration. The present invention provides a device that makes use of this principle and provides for the isolation of tumor cells from a large volume of blood.

By coupling the antibodies to ferrofluids (small magnetic particles) one can apply magnetic forces to contain the ferrofluids in a specific location while the suspension containing the cells is not contained. Different designs that incorporate the general principle can be understood from the present disclosure, providing for the realization of a device that can continuously capture target cells from cell suspensions within a large sample volume.

While not limiting the present invention to a specific design, four of the configurations are discussed below as preferred embodiments for the configuration of a ReFLECT device. These are described below in detail and proof of principle experiments have been conducted.

### Rolling Ferrofluids Configuration

One embodiment of the present invention, described as Rolling Ferrofluids, is represented in FIG. 2. FIG. 2 shows a device that contains a rotating disk with magnets of alternating orientation (8). The frequency of magnetic field alternation can be adjusted by the rotation speed of the disk, while the flood speed can be maintained using a peristaltic pump (not shown). The change in magnetic field direction makes the ferrofluid particles constantly turn in order to align themselves to the changing field. With the right frequency of alternation, this turning motion becomes a rolling movement. Ferrofluids are moved from the outlet to the inlet. In this design, a fluid containing ferrofluids (9) is present in a loop along the rotating magnets (8). At the inlet (10), blood or DLA product enters the loop and is brought into contact with the ferrofluids. At the point where the loop deviates from the rotating disk (11), the ferrofluids or DLA product are mixed and the ferrofluids bind to the CTC. The mixture is incubated in the incubation volume (12).

When the tubing is again passed along the rotating magnets (13) the ferrofuids and CTC-bound ferrofluids will move towards the magnets. At the position where the tubing loses contact with the magnets (see FIG. 2 insert), the CTC (14) and ferrofluids (15) will be contained along the rotating magnets while the DLA product is re-circulated. The ferrofluid thus remains within the cartridge resulting in a continuous effective capture of CTC. The blood or DLA product void of CTC and ferrofluids can be discarded or recirculated through the outlet (16). In this configuration the inlet (10) and outline (16) could also be connected directly to the patient's vascular system.

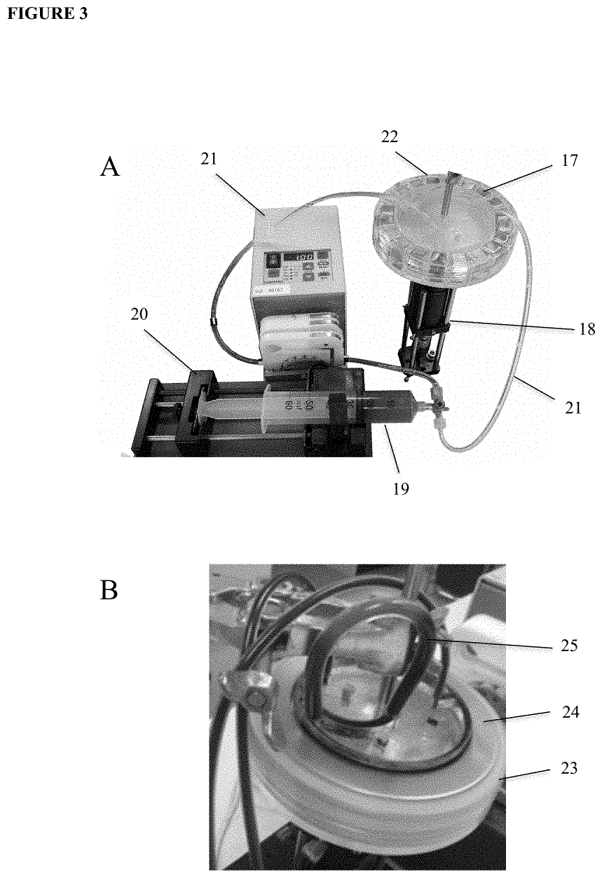

A device was designed and built according to the Rolling Ferrofluids device concept and is illustrated in FIG. 3. The device in FIG. 3A consists of rotating magnets (17), a motor to rotate the magnets (18), a syringe containing the sample from which CTC are to be removed (19), a syringe pump (20), the tubing to pass the sample (21), and the incubation chamber and ferrofluid loop (22). The latter is shown in more detail in FIG. 3B. The magnets are now being rotated (23), the ferrofluid loop containing ferrofluids and captured cells (24) and incubation chamber (25) can be seen in FIG. 3B. The device was used to demonstrate the feasibility of the present invention in sampling a large volume. Using the Rolling Ferrofluid concept in the device set-up in FIG. 3 it was demonstrated that:
Ferrofluids can be retained and re-circulated without leakage with a flow speed of 2 ml/min.
Ferrofluids could be continuously re-circulated within the device for >8 hours
Breast cancer cells from the cell line SKBR-3 in a saline solution could be captured in a single pass with an efficiency of 93%-99%.
SKBR-3 cells spiked in blood could be captured in a single pass with an efficiency of 30%.

Additionally, it was shown that ferrofluids, used to process a blood sample from a healthy donor on the CellSearch system, could be used to capture MCF-7 cells spiked in healthy donor blood without loss of efficiency.

### Flow Switching Configuration

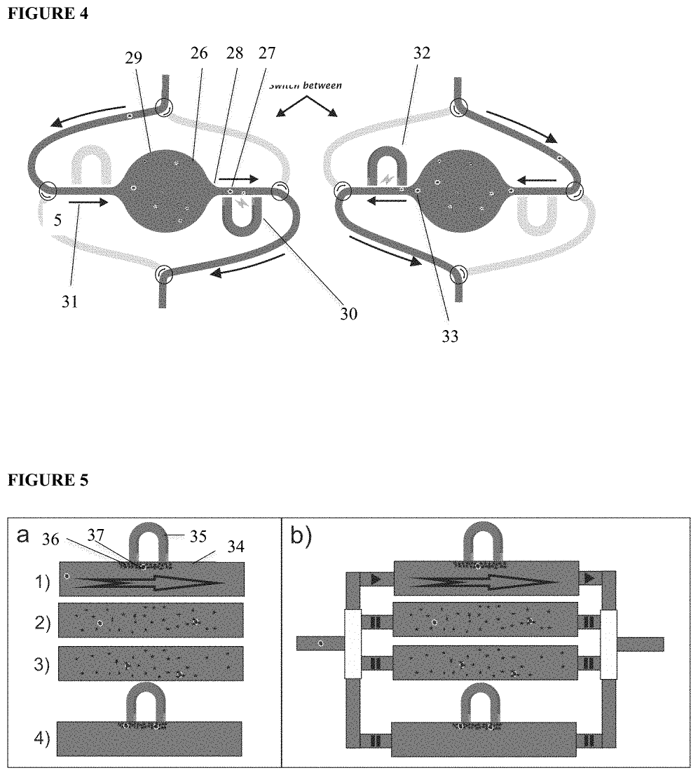

FIG. 4 depicts a schematic representation of a further embodiment of the present invention and is based on the switching of the flow direction. The principle of this embodiment is to first capture ferrofluids (26) and bound tumor cells (27) at the outlet (28) of the incubation chamber (29). Next, by moving the magnets (30) to the other side of the chamber (31) and reversing the flow direction (32) across the incubation chamber, the ferrofluids are flushed back into the incubation chamber. The side of the incubation chamber where the magnet has moved has now become the outlet (33), and ferrofluids and tumor cells will start to accumulate on this end. Switching between these two configurations allows the ferrofluids to repeatedly move back and forth across the incubation chamber allowing continuous capture of CTC.

### Chamber Containment Configuration

A still further embodiment is illustrated in FIG. 5, Panel (a) and comprises a chamber (34) in which ferrofluids are exposed to an (electro-)magnet (35) positioned below the chamber.

The four stages are described below and shown in FIG. 5a:
1. The magnet is on (present) and ferrofluids from a first volume are pressed against the bottom of the chamber. A second volume of blood or DLA product is allowed to flow into the chamber while the magnet keeps the ferrofluids (36) in the chamber.
2. Once the chamber is flushed with a second volume, the magnet is turned off (removed) to allow the ferrofluid to mix and bind to the tumor cells (37) present in the second volume. The process may optionally have a magnetic mixing step.
3. The ferrofluid and blood cells are given time to incubate, thereby ensuring that tumor cells can bind enough ferrofluid to be attracted to the magnet.
4. The magnet is once again turned on and the ferrofluid and tumor cells are pulled to the bottom of the chamber, resulting in the same situation as at the start of the cycle with the first volume.

As the four stages form a cycle, the process can be repeated continuously in a way similar to intermittent flow centrifugation used in leukapheresis. Alternatively, it is possible to switch the fluid flow between four or more identical chambers, each in a different stage of the process as shown in FIG. 5 Panel (b).

### Curtain Configuration

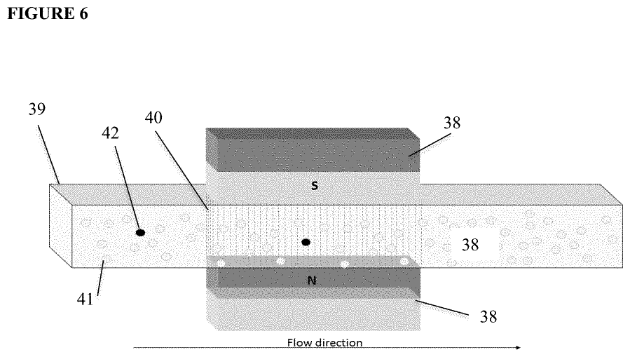

A still further embodiment is represented in FIG. 6. A magnetic field is created by placement of magnets (38) on two opposite sides of a flow chamber (39) such that ferrofluids (40) introduced into the flow chamber will align between the opposing magnets. The ferrofluids are coated with ligands for which a binding pair is present on the targets cells. After ferrofluids contained in a fluid are passed through the flow chamber they will form a curtain throughout the part of the flow chamber that is in between the magnets. The magnets are positioned during the filling of the chamber such that the distribution of ferrofluids is obtained throughout the chamber and is optimal for the capture of cells passing through the chamber. After the curtain composed of ferrofluids is established, a fluid containing non-target cells (41) and target cells (42) is passed through the flow chamber. Whereas non-target cells will pass through the chamber, the target cells will bind to the ferrofluids. After the fluid has been passed the magnets can be removed and the ferrofluids with the target cells retrieved from the chamber. The dimensions of the chamber, the concentration of ferrofluids and magnetic field can be optimized such that an optimal target cell capture is obtained with the least amount of capture of non-target cells.

Isolation, Identification and Characterization of CTC from ReFLECT

In all embodiments disclosed, the cells captured by ReFLECT are released, identified and characterized for the presence or absence of treatment targets. As in all concepts a magnetic force keeps the CTC and ferrofluids in the device, extraction will in all cases take place by removing the magnet(s) and flushing the cartridge. The volume containing ferrofluids and CTC will need to be reduced in order visualize and identify the CTC, after which characterization on the single cell level is needed to investigate the heterogeneity of the CTC population.

While all known means for the isolation, identification and characterization of CTC are considered in the present invention, one option is to utilize the VyCAP single cell analysis platform, see U.S. Pat. No. 9,638,636 issued 2 May 2017. This platform comprises two parts and is discussed below: seeding and isolation.

### Seeding

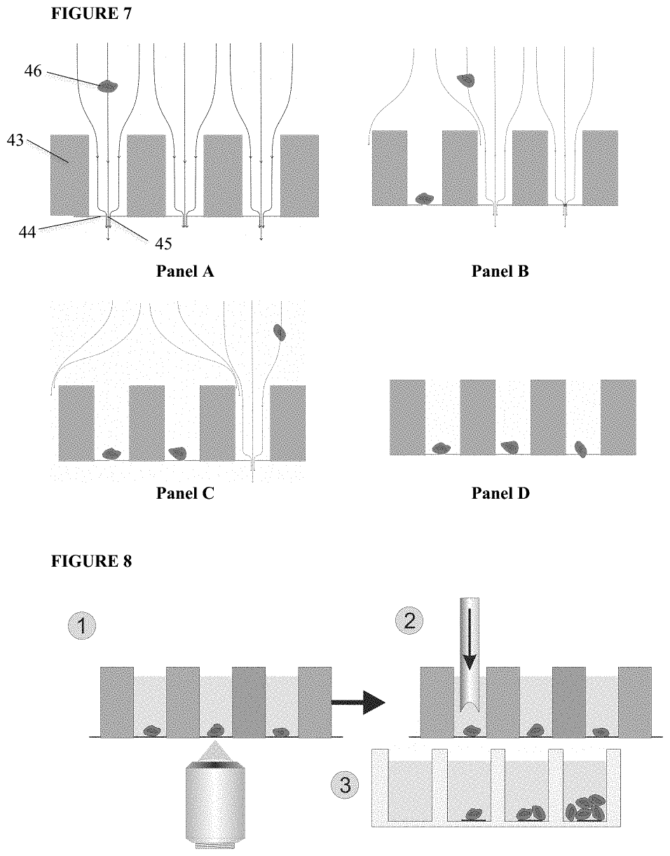

The solution with CTC and ferrofluids is extracted from the ReFLECT cartridge and placed on a silicon chip comprising 6400 microwells. As shown in FIG. 7, each microwell is a silicon wafer, (43), having a diameter of 70 μm and a height of 360 μm. The bottom of the well is a 1 μm thin SiN layer, (44) with a single pore, (45). By applying a small vacuum pressure the cell suspension fluid enters the well and exits through the pore in the bottom, hereby dragging the cells along. Once a cell has landed onto the pore, the flow through that particular well stops, and no other cell will enter. The remaining fluid and cells will be diverted to the next available microwell, resulting in a fast distribution of single cells into individual microwells.

A schematic illustration for seeding single cells into individual wells in the microwell platform is shown in FIG. 7. A sample fluid containing target events/cells, in this case a sample fluid with cells, (46) is added to the sample supply side, corresponding to the side with the large cavities in the microsieve. The fluid flows into each of the wells and flows out of the well through a single pore at the bottom plate of the membrane. Each well has a single pore with dimensions smaller than the objects of interest. The objects of interest are dragged by flow and hydrodynamic forces into the well, FIG. 7, Panel A. Consequently, the objects of interest will land on the pore of a well significantly restricting or stopping the flow rate through the pores, thereby minimizing the chance that a second object will enter the same well, FIG. 7 Panel B. This process continues as shown in FIG. 7, Panel C until all the sample fluid has passed through the wells. The end result is that each occupied well will contain one single cell, represented in FIG. 7, Panel D.

After adding seeding reagents to fluorescently label the cells, they are placed on top of the microwell plate. For identification of viable CTC, EpCAM (not cross blocking with VU1D9), CD45 (leukocytes), Calcein AM green (alive) and EthD1 red (dead) are used. Next, the slide with the microwell chip is transferred to an automated fluorescence scanning microscope. Fluorescence images of each of the single cells are acquired, FIG. 8, Panel 1. Based on the acquired images, the cells of interest are selected for isolation.

### Isolation

To isolate the single cells, a solid punch needle is lowered into the microwell that contains the cell to be isolated and punches out the SiN bottom together with the cell for collection, FIG. 8 Panel 2, in a reaction tube such as, but not limited to, Eppendorf tubes for DNA analysis or into the well of a culture plate for clonal expansion, see FIG. 8 Panel 3.

One application contemplated by the inventors of the present invention is the use of ReFLECT in a patient's blood stream in order to continually monitor the status of the patients CTC. This would be especially important before surgery for the removal of cancer where ReFLECT will determine whether or not the disease is disseminated and whether appropriate systemic treatment is needed along with the surgery. In this model, the device resembles a wrist watch, or cancer watch, such that when connected to the patient the watch captures CTC in a cartridge for further analysis. When the cartridge becomes full, the patient is notified to remove the cartridge for in-depth analysis of captured CTC and a new cartridge is inserted into the device, thus providing a means for tailoring the treatment of the disease based on the analysis. It is especially useful in metastatic disease and the determination of the spread of the disease. The inventors have preliminary evidence that the presence of CTC in this setting is indicative of relapse.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope thereof. Therefore, it is intended that the present disclosure not be limited to the particular embodiment(s) disclosed, but that the disclosure will include all embodiments falling within the scope of the present disclosure.

1- A device for capturing a target cell population in a biological fluid comprising:
a. a container having an incubation chamber with an inlet and outlet for continuous flow of the biological fluid containing a target cell population;
b. a fixed amount of unbound cell specific antibody-labeled ferrofluids contained in the incubation chamber; and
c. a magnetic field to position the unbound cell specific antibody-labeled ferrofluid for binding to the target cell population,
wherein the unbound and bound cell specific antibody-labeled ferrofluid is diverted from the continuous flow of the biological fluid at the outlet for the capture of the target cell population.

2- The device of claim 1 further having a recirculation means to recirculate the unbound cell specific antibody-labeled ferrofluid and continuously capture the target cell population.

3- The device of claim 2, where the target cell population in a biological fluid is circulating tumor cells in blood.

4- The device of claim 2, where the target cell population in a biological fluid is circulating tumor cells in diagnostic leukapheresis fluid.

5- The device of claim 1, where the container is a disposable cassette.

6- The device of claim 1, where the cell specific antibody is EpCAM.

7- The device of claim 1, where the magnetic field is from a rotating disk having magnets with alternating orientation whereby the unbound cell specific antibody-labeled ferrofluids have a rolling movement within the incubation chamber for continuous capture of the target cell population.

8- The device of claim 1, where the incubation chamber comprises:
a. the magnetic field at the outlet of the incubation chamber to capture the unbound and bound cell specific antibody-labeled ferrofluid;
b. a means for moving the magnetic field to the inlet side of the incubation chamber; and
c. a means for reversing the flow of the biological fluid in the incubation chamber towards the inlet.

9- The device of claim 1, where the magnetic field is an electro-magnetic field having a switching means where activating the magnetic field holds the unbound and bound cell specific antibody-labeled ferrofluid to an inner surface of the incubation chamber during flow and deactivating the magnetic field when the flow is stopped releases unbound and bound cell specific antibody-labeled ferrofluid to allow mixing, wherein repeated activation and deactivation causes the continuous capture of the target cell population.

10- The device of claim 1, where the incubation chamber comprises multiple incubation chambers having a valve to control flow of the biological fluid through each incubation chamber.

11- The device of any of claim 7, where magnets on opposite sides of the incubation chamber form the magnetic field for aligning the cell specific antibody-labeled ferrofluid in a curtain throughout the incubation chamber such that the target cell population is captured with the continuous flow of the biological fluid through the incubation chamber.

12- The device of claim 1, further comprising a means for interrogating the captured target cell population.

13- The device of claim 12 comprising:
a. self-seeding micro wells for isolating individual cells from the target population; and
b. micro well culture plate for analysis of the isolated individual target cells from the target cell population.

14- The device of claim 13 further having a solid punch needle to punch an individual target cell from the self-seeding micro well to the micro well culture plate.

15- A method for capturing cells from a target population in a biological fluid sample comprising:
a. continuously flowing a biological fluid through an incubation chamber having an inlet and outlet;
b. exposing a fixed amount of unbound cell specific antibody-labeled ferrofluid in the incubation chamber to the biological fluid containing the target population;
c. positioning the unbound cell specific antibody-labeled ferrofluid with a magnetic field to bind to the target cell population in the biological fluid; and
d. diverting the unbound and bound cell specific antibody-labeled ferrofluid from the continuous flow to separate cells from the biological fluid.

16- The method of claim 15 further recirculating unbound cell specific antibody-labeled ferrofluid to continuously capture the target cell population

17- The method of claim 15, where the target cell population in a biological fluid is CTC in blood.

18- The method of claim 15, where the target cell population in a biological fluid is CTC in diagnostic leukapheresis fluid.

19- The method of 15, where cell specific antibody is EpCAM.

20- The method of claim 15, where positioning the magnetic field is by a rotating disk having magnets with alternating orientation wherein the unbound cell specific antibody-labeled ferrofluids have a rolling movement within the incubation chamber for capturing target cell population

21- The method of claim 20, where positioning the magnetic field comprises:
a. exposing the unbound and bound cell specific antibody-labeled ferrofluid to a magnetic field at the outlet of the incubation chamber;
b. moving the magnetic field to the inlet side of the incubation chamber; and
c. reversing the flow of the biological fluid in the incubation chamber towards the inlet; and
d. repeating steps (a), (b), and (c).

22- The method of claim 21, where positioning the magnetic field comprises:
a. activating an electro-magnetic field to hold the unbound and bound cell specific antibody-labeled ferrofluid to an inner surface of the incubation chamber during flow; and
b. deactivating the electro-magnetic field to allow mixing of the unbound cell specific antibody-labeled ferrofluid with the target cell population,
wherein repeating steps (a) and (b) causes the continuous capture of the target cell population.

23- The method of claim 20, where positioning the magnetic field comprises:
a. orienting magnets on opposite sides of the incubation chamber wherein the cell specific antibody-labeled ferrofluids align as a curtain throughout the incubation chamber;
b. capturing the target cell population with the continuous flow of the biological fluid through the incubation chamber.

24- The method of claim 15, further comprising interrogating the captured target cell population.

25- The method of claim 15 comprising:
a. isolating individual cells from the target population in a self-seeding micro well; and
b. analyzing isolated individual cells from the target population.

26- The method of claim 25 where seeding reagents are added to isolated individual cells to fluorescently label the isolated individual cells.

27- The method of claim 26, where DNA or RNA is analyzed.

28- The method of claim 27, where isolated individual cells are analyzed by clonal expansion.

29- The method of claim 25, where the isolated individual cells are analyzed for heterogeneous subsets.

30- The method of claim 29 where analysis of the heterogeneous subsets is used to treat a subject having cancer.

31- A method for analyzing the heterogeneity of a CTC population in a biological fluid sample comprising:
a. continuously flowing a biological fluid through an incubation chamber having an inlet and outlet;
b. exposing a fixed amount of unbound CTC specific antibody-labeled ferrofluid in the incubation chamber to the biological fluid containing the CTC population;
c. positioning the unbound CTC specific antibody-labeled ferrofluid with a magnetic field to bind to the CTC cell population in the biological fluid;
d. diverting the unbound and bound CTC specific antibody-labeled ferrofluid from the continuous flow to separate cells from the biological fluid,
wherein the continuous flow of the biological fluid containing the CTC population is an individual patient.
